(19) United States
(12) Patent Application Publication
(10) Pub. No.: US 2010/0087748 A1
(43) Pub. Date: Apr. 8, 2010

(54) DEVICE AND METHOD FOR SENSING RESPIRATION OF A LIVING BEING

(76) Inventors: Andreas Tobola, Hemhofen (DE); Sergev Ershov, Erlangen (DE); Uwe Wissendheit, Erlangen (DE); Robert Couronne, Erlangen (DE)

Correspondence Address: GLENN PATENT GROUP, 3475 EDISON WAY, SUITE L, MENLO PARK, CA, 94025, US

(21) Appl. No.: 12/574,603

(22) Filed: Oct. 6, 2009

(30) Foreign Application Priority Data
Oct. 7, 2008 (DE) 102008050640.0
Nov. 6, 2008 (DE) 102008056252.1

(51) Int. Cl. A61B 5/08

(52) U.S. Cl. 600/529

(57) ABSTRACT

A description is given of a device for sensing respiration of a living being, the device including: an active transmitter configured to generate a magnetic or electromagnetic field; and a sensor arranged on the torso of the living being and configured to provide a signal which depends on the magnetic or electromagnetic field and on a change in the distance, caused by the respiration of the living being, between the active transmitter and the sensor.

### CROSS-REFERENCE TO RELATED APPLICATIONS

This application claims priority from German Patent Application No. 102008050640.0, which was filed on Oct. 7, 2008, and claims priority from German Patent Application No. 102008056252.1, which was filed on Nov. 6, 2008, and which are incorporated herein by references in their entirety.

The present invention relates to devices and methods for sensing respiration or a respiration activity on the part of living beings, e.g. humans or animals.

### BACKGROUND OF THE INVENTION

What is referred to as respiration activity is the variation in the body's circumference at at least one location on the torso, e.g. on the rib cage, of a living being. Typically, with human beings, respiration activity is measured at two locations on the torso, at the level of the abdomen and the torso, and is mapped as a signal or stored.

From the raw signal of respiration activity, vital parameters such as breathing rate, breathing amplitude and breathing volume are calculated, which provide valuable information about the condition of the person. Generally, a statement may be made about the condition of a person or patient by means of respiration activity, either on its own or in combination with other vital parameters.

Conventional methods are based on measuring respiration activity by means of straps placed around a body part. The method by means of which respiration activity is determined may be classified into two categories. On the one hand, these are no-load methods and, on the other hand, they are methods which involve a certain amount of tension of the straps. An example of a no-load method is inductive plethysmography, wherein the straps are loosely placed around the selected body parts. Irrespective of the method of measurement, such straps may be worn underneath the clothing, but may be connected, underneath the clothing, to signal processing electronics underneath the clothing via cables.

U.S. Pat. No. 5,825,293 describes a method of monitoring the breathing rate by means of detecting the changes in the magnetic field of a permanent magnet attached to the body of the patient.

A disadvantage of respiration straps is that they may be placed on the body and be connected to signal processing electronics prior to measurement.

A disadvantage of the method wherein the magnets are integrated into a piece of clothing is that the respective patient has to wear such a piece of clothing provided with a magnet, or may put it on beforehand.

### SUMMARY

According to an embodiment, a device for sensing respiration of a living being may have: an active transmitter configured to generate a magnetic or an electromagnetic field; and a sensor arranged on the torso of the living being and configured to provide a signal which depends on the magnetic or electromagnetic field and on a change in the distance, caused by the respiration of the living being, between the active transmitter and the sensor.

According to another embodiment, a method of sensing respiration of a living being may have the steps of generating a magnetic or electromagnetic field by means of an active transmitter; and providing a signal by means of a sensor arranged on the torso of a living being, the signal depending on the magnetic or electromagnetic field and on a change in the distance, caused by the respiration of the living being, between the active transmitter and the sensor.

According to another embodiment, a measuring system for sensing respiration of a person situated within a motor vehicle may have: an active transmitter integrated into a backrest of a car seat of the motor vehicle and configured to generate a magnetic or electromagnetic field; and a sensor arranged within a safety belt of the motor vehicle, in a state in which the safety belt is fastened, at the height of the person's torso, and configured to provide a signal which depends on the magnetic or electromagnetic field and on a change in the distance, caused by the respiration of the living being, between the active transmitter and the sensor.

Embodiments of the method and of the device enable performing measurements of respiration activity with reduced impairment of the person or, generally, of a living being.

In embodiments of the method and of the device, the devices for measuring respiration activity, the active transmitter and the sensor are integrated into environment objects with which a person or, generally, a living being comes into direct contact. Examples of such environment objects are the backrest of a car seat, and the safety belt.

Embodiments of the device and of the method do not presuppose that so-called application parts, i.e. parts which are mounted or attached to the patient's body (e.g. respiration straps), be placed around the body for measuring respiration activity.

Embodiments of the device and of the method may be readily integrated into so-called “environment objects” of the person, e.g. operating elements, seats, beds, safety belts, steering wheels, etc., such that from the outside, they are inconspicuous or cannot be seen. Thus, the measurements of the person or the living being may be performed without being noticed or without involving any additional effort on the part of the person, such as placing the respiration straps or putting on corresponding pieces of clothing.

Embodiments of the device and of the method may further be used in such places or cases where respiration straps or specific pieces of clothing cannot be used because of hygienic and technical problems or because of non-acceptance by patients.

In embodiments of the present invention, the active transmitter is configured to adapt the device for sensing to varying conditions of measurement by changing, for example, the frequency or amplitude of the magnetic or electromagnetic field. For example, the transmitting power may be increased or reduced, depending on the average distance between the active transmitter and the sensor, which is a measure of the thickness of the torso, or rib cage, so as to enable reliable measurement but at the same time to keep the power consumption low, for example. Thus, optimum measurement is enabled irrespective of the circumference of the torso of the living being.

In further embodiments of the device and the method, wherein not all of the components of the measuring system or of the device can be integrated into environment objects of the person or other living beings, individual components of the device for sensing may be attached to the body of the person or living being. This is still simpler and faster than utilizing the conventional respiration straps, for example.

### BRIEF DESCRIPTION OF THE DRAWINGS

Embodiments of the present invention will be detailed subsequently referring to the appended drawings, in which:

Embodiments of the present invention will be explained below in more detail with reference to the accompanying figures, wherein:

FIG. 1a shows a schematic drawing of an embodiment of a device for sensing respiration of a living being;

FIG. 1b shows an embodiment, in accordance with FIG. 1a, wherein the active transmitter and the sensor are inductively coupled;

FIG. 2 shows a block diagram of an active transmitter of an embodiment of a device for sensing respiration of a living being;

FIG. 3 shows a block diagram of an embodiment of a sensor or a measuring/receiving device of a device for sensing respiration of a living being;

FIG. 4 shows a schematic representation of an embodiment of a device for sensing respiration of a living being, said device being integrated into a backrest of a car seat and into a safety belt, the measurement signal being communicated to the evaluation means via a wired connection;

FIG. 5 shows, at the top, a block diagram of an embodiment of an active transmitter and, at the bottom, a block diagram of an embodiment of a sensor of an embodiment in accordance with FIG. 4;

FIG. 6a shows a schematic representation of an embodiment of a device for sensing respiration of a living being, said device being integrated into a backrest of a car seat and into a safety belt, the measurement signal being communicated to the evaluation means via a wireless interface;

FIG. 6b shows a schematic representation of an embodiment of a safety belt and of a sensor for wireless transmission of the measurement signals;

FIG. 7 shows, at the top, a block diagram of an active transmitter and, at the bottom, a block diagram of a sensor for utilization in an embodiment in accordance with FIG. 6a;

FIG. 8a shows a schematic representation of an embodiment of a device for sensing respiration of a living being, said device being integrated into a backrest of a car seat and into a safety belt, the wireless transmission of the measurement signals being effected by means of load modulation or back-scattering methods;

FIG. 8b shows a schematic representation of an embodiment of a safety belt comprising an integrated transponder as a sensor;

FIG. 9 shows, at the top, a block diagram of an embodiment of an active transmitter and, at the bottom, a block diagram of an embodiment of a sensor for utilization in a device in accordance with FIG. 8a;

FIG. 10 shows, at the top, a further embodiment of a device for sensing respiration of a living being, wherein the active transmitter is attached to a bed or a lying surface, and the sensor is attached to a belt which is connected to the bed or the lying surface, and FIG. 10 shows, at the bottom, a further embodiment of a device for sensing respiration of a living being, wherein the sensor is integrated into a cushion which may be placed upon the torso of the living being;

FIG. 11 shows a measured breathing curve over several seconds;

FIG. 12 shows an embodiment of a sensor for inductive coupling; and

FIG. 13 shows an embodiment of a device for sensing respiration of a living being, said device being integrated into the backrest of a car seat and into a safety belt.

The term measuring system may also be used for the term device for sensing, the terms transmitter device or transmitter unit may also be used for the term active transmitter, the terms receiver device or receiver unit may also be used for the term sensor, the terms first and/or second object or environment object may also be used for the first and/or second means.

### DETAILED DESCRIPTION OF THE INVENTION

In the following, identical reference numerals will be used for objects and functional units which have identical or similar functional properties, repeated descriptions of said objects and functional units being dispensed with in order to avoid unnecessary repetitions.

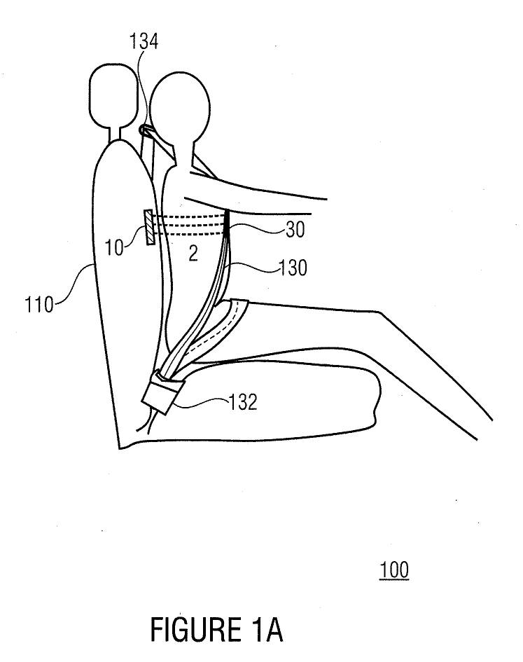

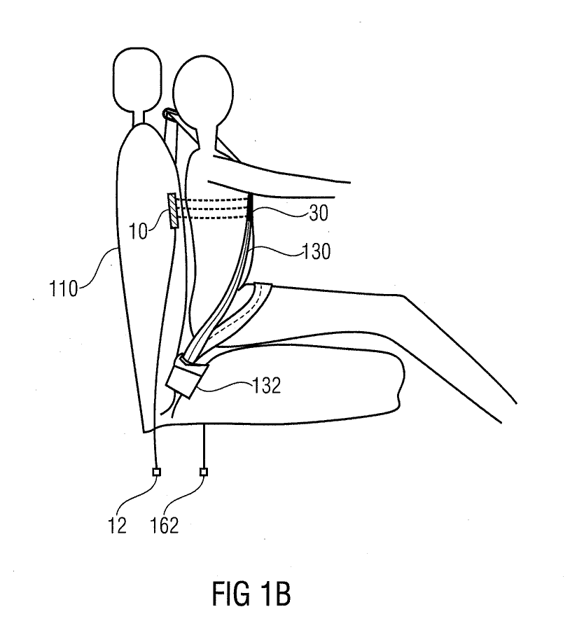

FIG. 1a shows a schematic representation of an embodiment of a device 100 for sensing respiration of a living being, in this case of a person, said device being integrated into an automotive environment.

The device 100 for sensing comprises an active transmitter 10 and a sensor 30, the active transmitter being configured to generate a magnetic or electromagnetic field, and the sensor being configured to provide a signal which depends on the magnetic or electromagnetic field and on a change in the distance, caused by the respiration of the living being, or person, between the active transmitter 10 and the sensor 30. In this context, the sensor 30 is arranged on the torso of the living being.

In the embodiment shown in FIG. 1a, the active transmitter 10 and the sensor 30 are arranged on opposite sides of the person's upper body 2.

Further embodiments (see FIG. 1a) additionally comprise a first means 110 and a second means 130, the first and second means being configured and connected to each other such that the active transmitter 10 is arranged within or on the first means 110, and the sensor 30 is arranged in or on the second means 130, such that the active transmitter 10 and the sensor 30 are arranged, or remain, on opposite sides of the torso irrespective of a turn or a turning movement of the torso 2 of the living being.

In the embodiment shown in FIG. 1a, the first means comprises a backrest of the car seat, and the second means comprises a safety belt 130. However, the backrest and the safety belt are only examples of first and second means, and embodiments are not limited thereto. In this context, as is shown in FIG. 1a, the active transmitter 10 may be arranged on or integrated within the first means 110, and the sensor 30 may be arranged on or integrated within the second means 130, e.g. the safety belt 130, or vice versa, the active transmitter 10 may be integrated within the second means 130, and the sensor 30 may be integrated within the first means 110.

In this context, the term “integrated” is generally used below irrespective of whether the active transmitter 10 and/or the sensor 30 are entirely or partly arranged within or outside of the first or second means 110, 130.

In further embodiments, the first or second means 110, 130 is a bed or a lying surface, for example, and the correspondingly other means may be a belt which may be connected to the bed or lying surface, or a cushion, for example, into which the active transmitter or the sensor is integrated.

FIG. 1a shows an embodiment of the device for sensing respiration, wherein the first means 110 and the second means 130 are connected to each other, in FIG. 1a by means of the belt fastener 132 and the belt, guide 134, which in turn are fixedly connected to the chassis of the vehicle (not shown) and, thus, also to the car seat, or to the backrest 110 of the car seat. The connection between the first means 110 and the second means 130 enables the person's torso 2 to turn, for example with regard to his/her longitudinal axis, between the first means 110 and the second means 130, and the active transmitter 10 and the sensor 30 are nevertheless located on opposite sides of the torso 2 and, thus, enable measurement of the change in the distance between the active transmitter 10 and the sensor 30 as a measure of respiration activity. To enable this movement, one of the two means, or both, may be elastic or elastically attached, cf., e.g., the safety belt comprising the elastic roll-up mechanics.

In further embodiments, a safety belt as the second means 130 is connected to the bed or the lying surface as the first means 110 so as to reliably measure a change in the distance, which is caused by the respiration, despite a turning of the torso.

Even though the embodiment in FIG. 1a shows a device comprising first and second means which are connected to each other, alternative embodiments may comprise first and second means which are not connected to each other or which comprise no first or second devices.

Generally, it may therefore be said that the device for sensing or for measuring respiration activity comprises an active transmitter 10, which is also referred to as a transmitter device, and a sensor, which will also be referred to as a receiver device 30 below. wherein the measurement is based on a change in the distance between the transmitter unit and the receiver unit and may therefore be sensed by means of metrology. The measurement signals within the receiver may be caused by different physical mutual influences between the transmitter unit and the receiver unit, e.g. by magnetic fields or electromagnetic fields.

What follows is a more detailed description of embodiments which are based on actively generating a magnetic field on the part of the transmitter device 10, i.e. on inductive coupling. In such embodiments, the device 100, which will also be generally referred to below as a measuring system, essentially consists of two coils which are loosely inductively coupled to each other. The primary coil may be integrated into an object, e.g. a car seat or an operating table, and generates an alternating magnetic field having a specified frequency, amplitude and/or phase position. For stabilizing purposes or for measuring interference effects or for measuring amplitude fluctuations, the transmitting unit and possibly also the receiving device may comprise additional measuring coils, or additional measuring coils may be integrated in the environment object.

For measuring respiration activity, a secondary coil or a secondary coil system is attached, as a sensor, at a specific distance from the primary coil or primary coil system of the active transmitter 10, to the object to be measured in such a manner that a change in the movement between the two coils or coil systems will be reflected in the form of a measured quantity. In embodiments based on inductive coupling, the measured quantity to be determined, or the signal to be generated, is a change in the induced voltage generated within the secondary coil, said change being due to the positional deviation of the secondary coil in relation to the primary coil. In this context, what is sensed and evaluated is not the induced voltage itself, but the change in the induced voltage, which is due to the positional deviation from a static position or starting position.

Data communication of the measurement signal thus generated may be effected in several ways: a) the measuring unit, or sensor, 30 may be wire-connected to the evaluation unit, b) the sensor 30 may be wirelessly connected to the evaluation unit, e.g. via Zigbee or Bluetooth (not shown in FIG. 1a), and c) the measuring unit 30 may transfer its data to the primary coil 10 by means of load modulation via a feedback of the secondary coil 30, so that the data signals can be detected there.

FIG. 1b shows an embodiment of a device for sensing respiration of a person, wherein the breathing rate of the person in the automobile is to be monitored. The active transmitter 10 and the sensor 30 are inductively coupled. The active transmitter 10 comprises a primary coil and possibly further reference coils integrated into the backrest of the car seat 110. The active transmitter 10 is configured to generate the magnetic field by means of a current fed into the primary coil. The sensor 30 comprises a secondary coil and is integrated into the safety belt 130. For example during the ride, or when the safety belt is fastened, the safety belt 130 is firmly applied to the driver's rib cage. By means of the inductive coupling, the magnetic flow generated by the primary coil flows through the secondary coil and in the process induces a voltage which may be increased even more by means of a resonant circuit with a parallel capacitor. By means of a subsequent demodulation circuit, it is not the induced voltage itself that is determined, but a change in the induced voltage. Thus, the driver's respiration activity may be monitored. If the driver is not breathing, the measuring circuit does not generate any measurement signal, or merely generates a “zero signal”.

As was set forth above, conventional respiration measuring systems involve applying so-called “application parts” (e.g. respiration straps) to the driver, but this is not necessary here. Once the person has got into the vehicle and once the safety belt has been fastened, measurement of respiration may start immediately. Thus, respiration monitoring even of changing drivers is readily possible. The system components and the supply lines may be designed such that they are inconspicuous or are even not visible from outside.

Sensing or measuring the signals which are a measure of the driver's respiration activity is possible during the ride, so that an evaluation of the condition of the driver of the vehicle by utilizing the quantities derived from the measurement signal or raw signal is also possible during the ride.

In the embodiment, shown in FIG. 1b, of the device for sensing respiration, the evaluation unit is wire-connected, see reference numeral 162 for the connection for tapping the sensor signal or measurement signal from the sensor 30. Reference numeral 12 designates the connection for the current supply of the active transmitter 10.

Alternative embodiments transfer the data from the sensor to the evaluation unit via radio transmission, load modulation, back-scattering methods or similar methods.

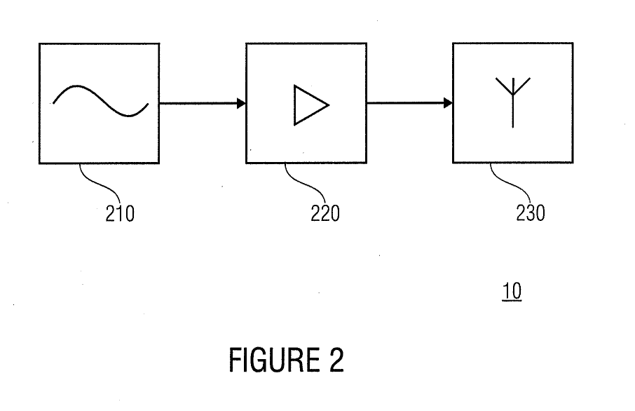

FIG. 2 shows a block diagram of an embodiment of an active transmitter 10 comprising a signal generator 210, an amplifier 220 and an antenna unit 230. The signal generator 210 is configured to feed a current into the primary antenna unit (see arrow), which comprises a primary coil and a matching network, via a signal (see arrow) applied to the amplifier 220, e.g. a power amplifier or an output stage, and thus to generate an alternating magnetic field having a specified frequency, amplitude and possibly a defined phase position. The signal generator 210 is a sinusoidal oscillator, for example.

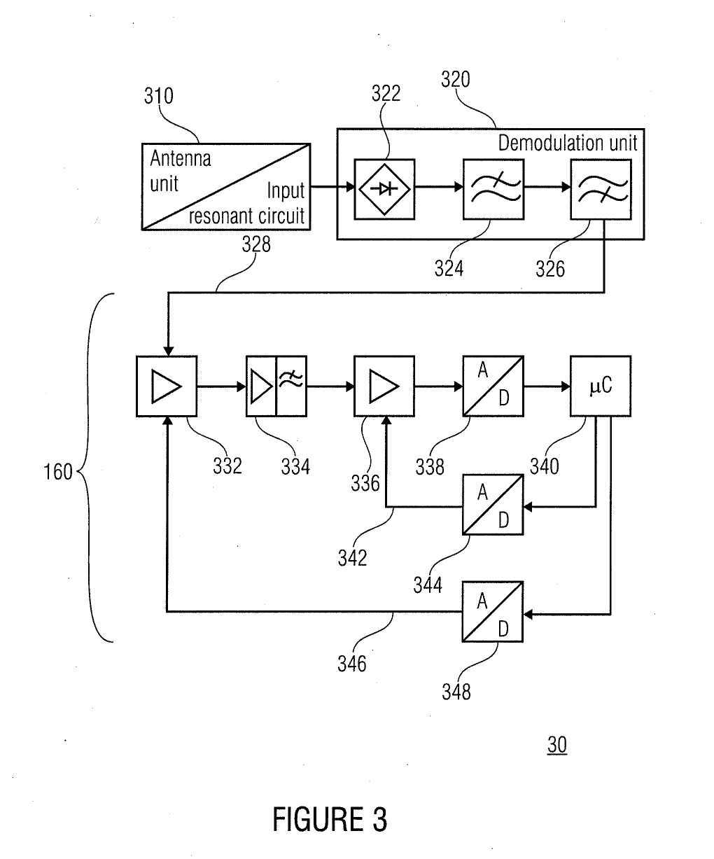

FIG. 3 shows a block diagram of an embodiment of a sensor or measuring device 30 comprising an antenna unit 310, a demodulation unit 320, and a signal processing unit 160. The sinusoidal signal of the primary coil 230 or of the active transmitter 10 of FIG. 2 induces a sinusoidal alternating voltage into the antenna unit 310 of the secondary side or of the sensor 30. The antenna unit 310 comprises, for example, a parallel resonant circuit including a matching network. The parallel resonant circuit, which comprises the secondary coil and a parallel capacitor, here serves to inductively couple the secondary coil and the primary coil of the active transmitter and to increase the voltage induced. The voltage change is detected by the subsequent demodulation unit 320. In a simple case, the demodulation unit 320 consists of a rectifier 322, which is connected upstream from two different filter stages 324 and 326. In this context, for example, the first filter stage may be a low-pass filter, and the second filter stage may be a high-pass filter or coupling capacitor (see FIG. 3). The rectifier 322 inverts negative signal portions of the voltage induced. The signal 328 at the output of the demodulation unit, which is received by the subsequent filtering (low-pass filtering 324 and separating-off the direct component 326) already contains the respiration signal.

FIG. 3 shows further amplifier, filter, matching and converter stages for further signal processing of the “respiration signal” 328, as well as a unit for controlling and evaluating the signals, here a microcontroller μC. In this context, reference numeral 332 designates an impedance converter, reference numeral 334 designates an amplifier having an active filter, reference numeral 336 designates an amplifier comprising “gain” control”, reference numeral 338 designates an analog/digital converter (AD converter), and 340 designates the microcontroller. The first feedback 342 from the microcontroller 340 to the amplifier having the gain regulation 336 serves to achieve gain control, whereas the second feedback 346 from the microcontroller 340 to the impedance converter 332 serves to achieve dynamic offset correction. Both the first feedback 342 and the second feedback 346 each comprise an AD converter 344 and 348, respectively. In further embodiments, an anti-aliasing filter may be implicitly used upstream from the analog/digital conversion 338, and a reconstruction filter may be used downstream from the digital/analog conversions 344, 348, which is not depicted in the figure for reasons of clarity.

The previously described embodiments related to signal changes, or changes in the signal 328, which are caused by a change in the distance between an active transmitter 10 and a sensor 30 which are inductively coupled to each other. Alternatively, embodiments may also comprise other coupling mechanisms instead of inductive coupling between the active transmitter 10 and the sensor 30, for example electromagnetic coupling.

The active transmitter 10, or the transmitter unit 10, of an embodiment comprising electromagnetic coupling includes, for example, a dipole or patch antenna which is supplied by a high-frequency signal. Signal generation itself is similar to the inductively coupled systems or embodiments. The same applies to embodiments of the receive antenna, the receive signal change of which, caused by a change in the field strength, is used for deriving respiration activity. Similarly to the active transmitter 10, the sensor 30 comprises dipole or patch antennas, for example.

Subsequently, the detected receive signal is further processed and evaluated in a manner similar to inductively coupled embodiments. In the case of embodiments having a transponder as the sensor, a back-scattering method is used instead of load modulation for transferring the measurement signal to the evaluation unit. Embodiments having a transponder as the sensor have the advantage that they make do without any additional power supply for operation on top of that for the active transmitter, but that the power supply is effected by means of the inductive or electromagnetic coupling.

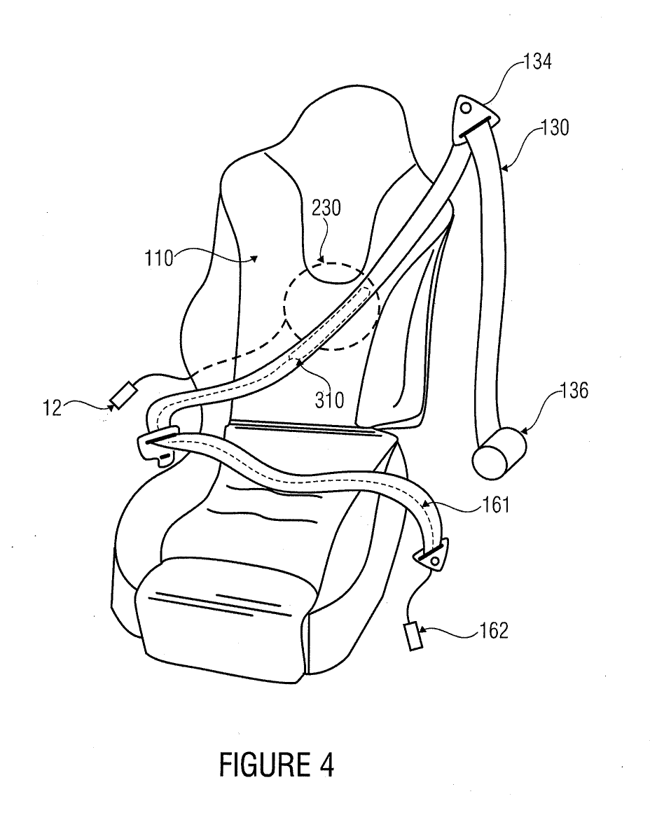

FIG. 4 shows a schematic representation of an embodiment of a device for sensing or monitoring a breathing rate of a person who is situated in an automobile, the measuring unit being wire-connected to the evaluation unit.

The device for sensing respiration of a person in accordance with FIG. 4 comprises a car seat, or the backrest of a car seat, as the first means 110, a safety belt as the second means 130, an active transmitter 10, and a sensor 30. To put it more precisely, FIG. 4 depicts the primary coil 230 of the active transmitter 10 (see dashed line) and the secondary coil 310 of the sensor 30 (see dashed line in the area of the belt). The first means 110 is connected to the second means 130 via the chassis, said second means 130 being connected to the same chassis via the belt guide 134 and the belt retractor 136, for example.

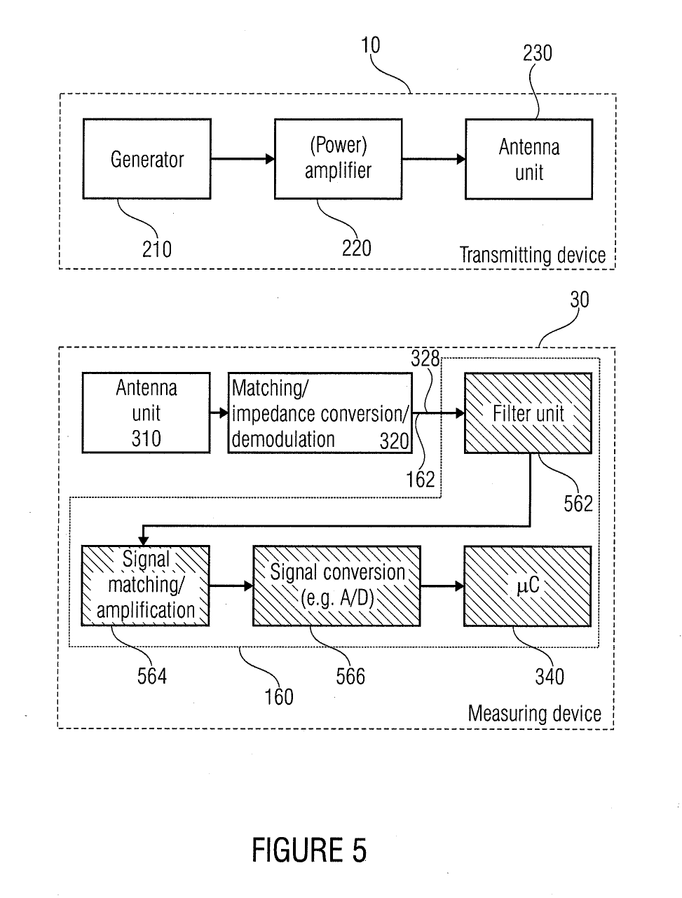

FIG. 4 shows an embodiment of an integration of a device for measuring respiration activity in automotive environments. The active transmitter, or the primary coil, 230 is integrated into the backrest 110 of the automobile seat so as to be opposite the driver's rib cage. By means of the connection 12, a sinusoidal signal which is generated from the outside and amplified, e.g. a sinusoidal current or voltage, is fed into the primary coil 230, and thus a magnetic field is generated at the antenna, i.e. at the primary coil 230. Further details of the active transmitter will be explained in more detail later on with regard to FIG. 5. Alternatively, the entire transmitter device 10, consisting of signal generation 210, signal amplification 220, and the transmit antenna 230, may be integrated directly into the backrest 110 of the automobile seat. In this case, there is no connection 12 to the outside.

The sensor 30, or the receiving device 30, comprises a secondary coil 310 as well as adaptation, impedance conversion and demodulation electronics 320. For example, the turns of the secondary coil 310 are woven into the safety belt 130 or are applied on a carrier film and subsequently glued or welded into the safety belt. The adaptation, impedance conversion and demodulation electronics is advantageously integrated, in immediate proximity to the secondary coil 310, into the safety belt 130, for example in the form of a small plastic enclosure (not depicted in the drawing), which is welded into the safety belt. The receiving device 30 is connected to the evaluation unit (not shown) via a line 161 (see dashed line in the belt) guided along the belt 130. For example, the line 161 may be directly woven into the safety belt, or it may be attached in the form of flat and thin wires along the safety belt. This line 161 extends from the receiving device 30 or, in other words, from the secondary coil 310 comprising the adaptation, impedance conversion and demodulation electronics 320, up to the bracing of the belt on the opposite side. Alternatively, the line 161 may be guided in the other direction along the safety belt up to the belt retractor 136. Both variants are possible from a technical point of view. However, the first variant is technically easier to implement. The line 161 ends with the connection 162 for tapping the sensor signal.

An embodiment of a measuring device 30 is depicted schematically at the bottom of FIG. 5. The measuring device, or sensor, 30 comprises the antenna unit 310, the adaptation, impedance conversion and demodulation unit 320, and the signal processing unit 160 (system units hatched in gray in FIG. 5, bordered with a dotted line). In accordance with an embodiment, the functional blocks or system units of the signal processing unit 160 are not integrated into the safety belt 130, but are accommodated in an external housing connected to the connection 162. As was already explained with reference to FIG. 3, for example, the signal processing unit 160 comprises a filter unit 562, a signal matching/amplification unit 564, a signal conversion unit, e.g. analog/digital conversion unit, 566, and a microcontroller 340.

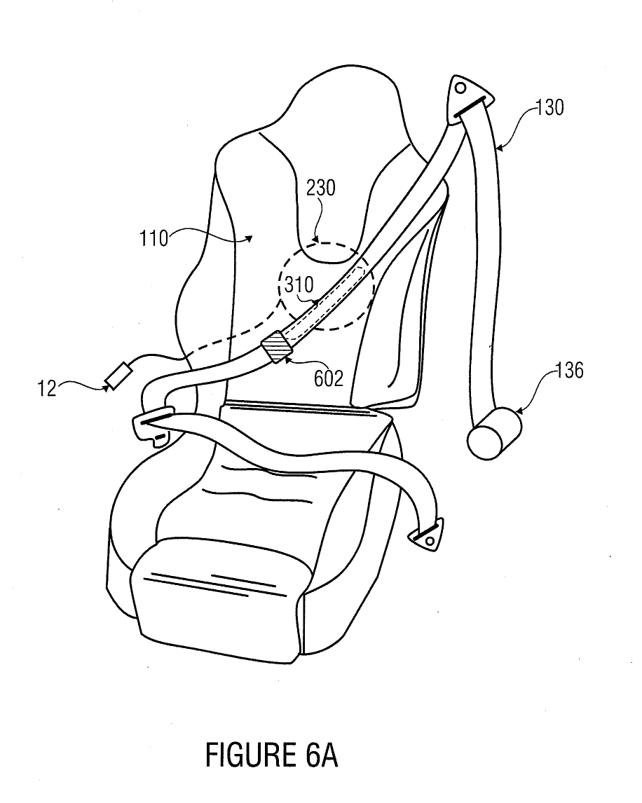

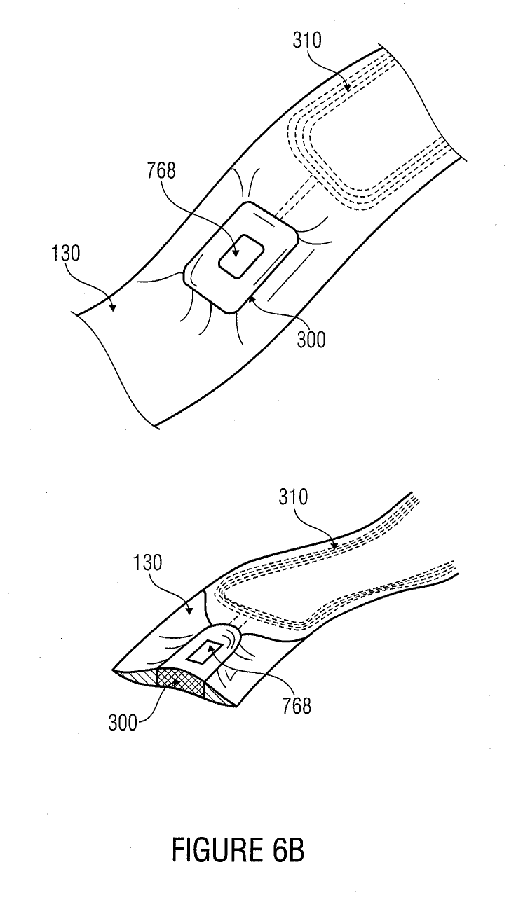

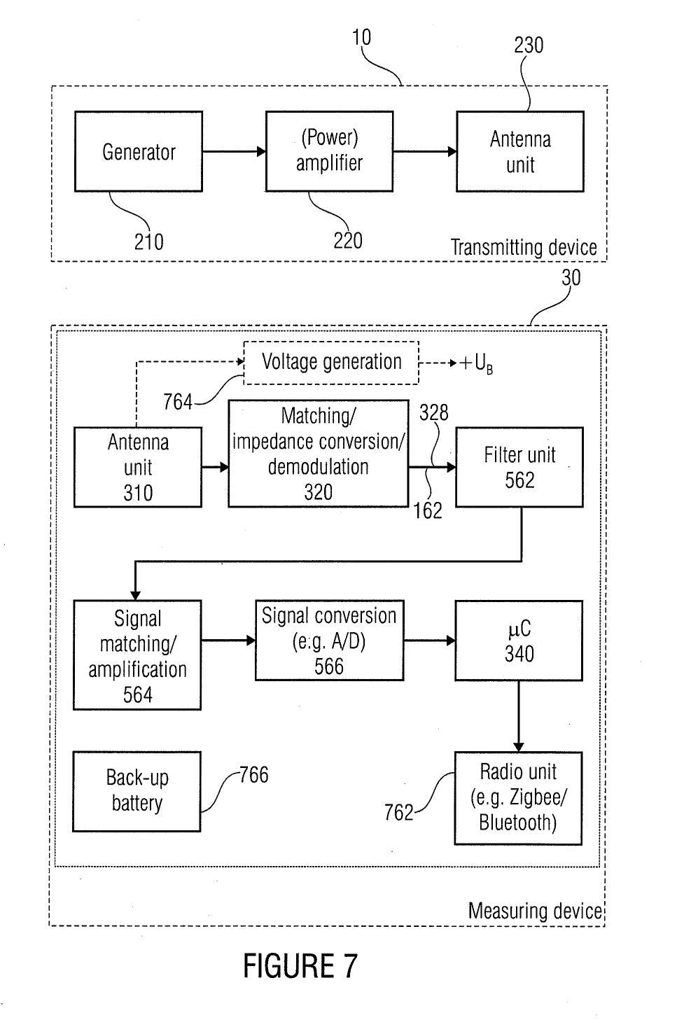

FIG. 6a shows a further embodiment of a device for monitoring the breathing rate of a person situated in an automobile, the measuring unit 30 being wirelessly coupled to the evaluation unit. FIGS. 6a, 6b show an embodiment of integrating the system for measuring respiration activity into an automotive environment similar to that shown in FIG. 4. The implementation of the transmitter device, or of the active transmitter, 10 (see top of FIG. 7) may be the same as that shown in FIG. 4, i.e. it does not differ from that of FIG. 4.

In this embodiment, the receiving or measuring device 30 additionally comprises the functions of data processing and wireless data communication, and it may be directly attached on the safety belt 130, for example in the form of an electronic data processing module in a small housing 602 (see FIG. 6a). The components or functional elements of the measuring device 30 are depicted at the bottom of FIG. 7. As compared to the measuring device in FIG. 5, the measuring device in FIG. 7 additionally comprises a radio unit 762, a voltage generation module 764, and possibly a back-up battery 766. By means of said units, the raw respiration signals 328 are sensed directly within the safety belt and are communicated to the evaluation unit via radio.

Therefore, in these embodiments, no further line from the receiving device 300 to the belt bracing or the belt retractor is necessary. The voltage generation module 764 may be selected to supply the entire electronic circuit of the receiving device 300 with power. In the event that the power consumption of the radio unit 762 exceeds the capacity of the voltage generation module 764, a back-up battery 766 may optionally be provided which may be replaced at any time via a battery compartment lid 768. The back-up battery may also be charged on the fly from the surrounding field using an integrated charging circuit, e.g. by means of a specific mode which is active whenever no measurements are conducted and, therefore, whenever the electronics make do with less energy.

FIG. 7 shows, at the top, a block diagram of the active transmitter 10, as was already described with regard to FIG. 5.

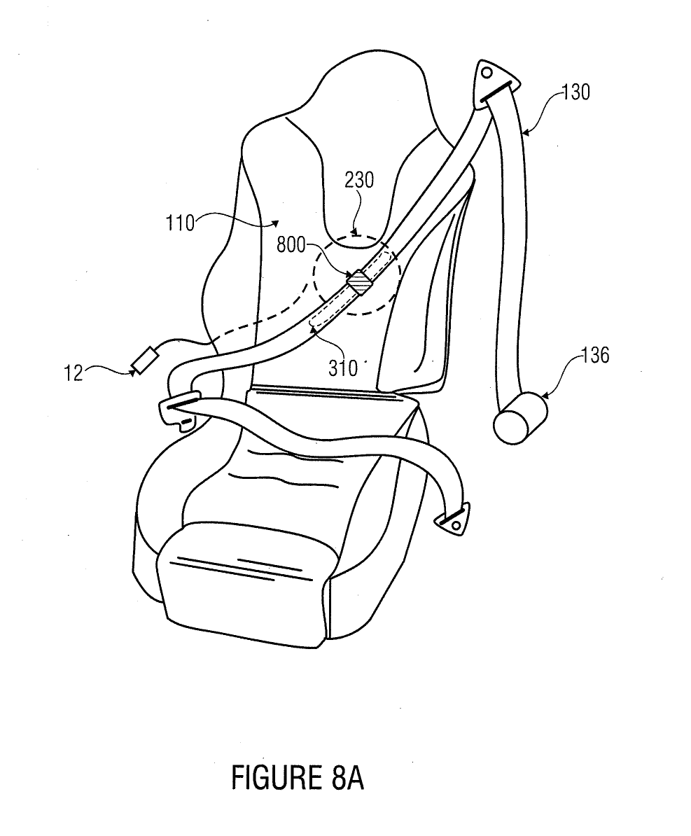

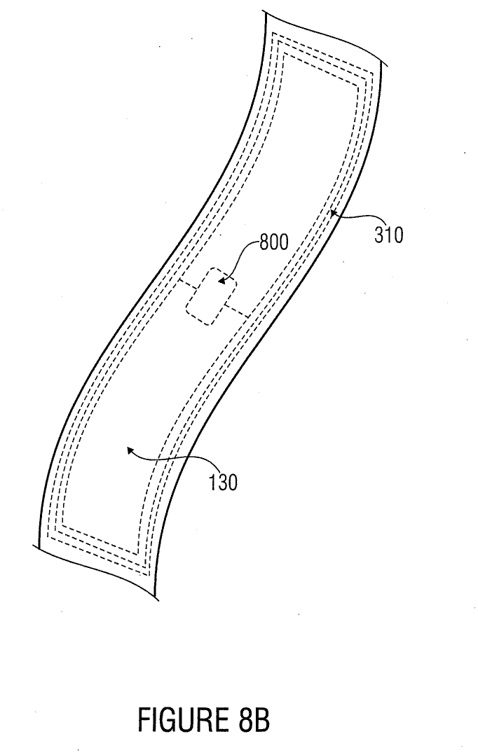

What follows is a description of a further embodiment of a device for monitoring the breathing rate of a person situated in an automobile, wherein data communication is effected by means of a feedback of the secondary coil to the primary coil by means of load modulation. FIGS. 8a and 8b show an embodiment of an integration of the system for measuring respiration activity into an automotive environment.

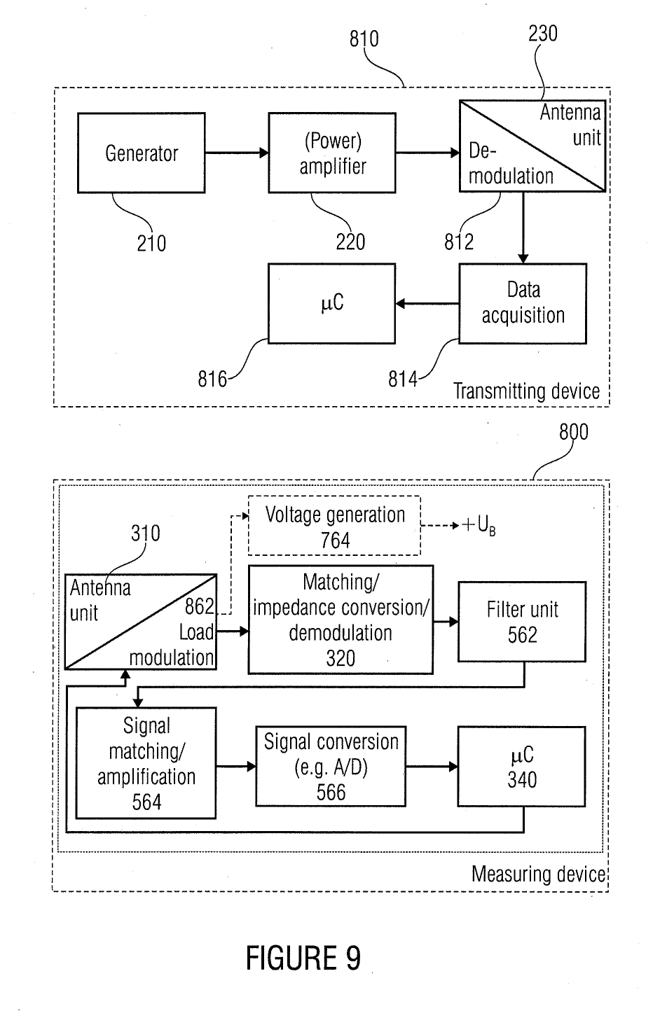

The transmitter device, or the active transmitter, 810 see the top of FIG. 9, differs from the transmitter device 10 of FIGS. 5 and 7 in that the transmitter device 810 additionally comprises a demodulation unit 812, a data acquisition unit 814, and a microcontroller 816. Said additional functional units serve to transmit data from the measuring means 800 to the transmitter device 810 by means of load modulation.

The receiving device or measuring device 800, see the bottom of FIG. 9, is integrated into the safety belt 130 and consists of the antenna unit 310, or the secondary coil 310, and the signal processing means 160. The turns of the secondary coil 310 are woven into the safety belt, and the measuring unit 800 is welded into the safety belt within a small plastic enclosure, for example, or is glued onto or into the safety belt, for example, so that from the outside, they are inconspicuous or cannot be seen. The measuring unit 800 is supplied by the induced voltage or the induced current obtained from the field. The measuring quantity, i.e. the changes in the induced voltages generated in the secondary coil, is formed by positional deviations of the secondary coil 310 from the primary coil 230. It directly correlates to the breathing movements in the ribcage. These changes in the induced voltages are detected, matched and converted to a digital signal by the electronics. This digital signal is transmitted to the primary coil 230 by means of the load modulation principle, i.e. the digitized respiration activity signal is transmitted to the transmitter device 810 by varying the coupling of the secondary coil 310 to the primary coil 230. To this end, the measuring device 800 comprises a load modulation unit 862 as the radio unit 762 in accordance with FIG. 7. The data is subsequently processed further within the transmitter device 810, and/or is forwarded to the evaluation unit by the connection 12.

Further embodiments of the device for sensing respiration, e.g. for monitoring the breathing rate, of a person placed in a functional bed or on an operating table will be described below.

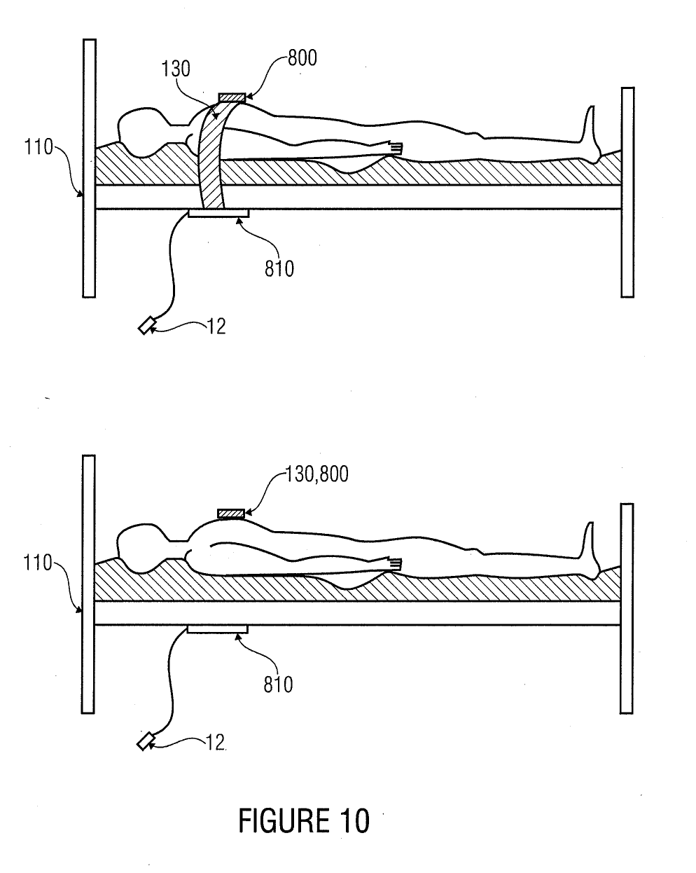

The top of FIG. 10 shows a functional bed or an operating table or the like as the first means 110, a patient fixation belt as the second means 130, the active transmitter 810 arranged on the underside of the functional bed 110, and the sensor or measuring device 800, which is integrated onto or into the belt 130, so that the active transmitter 810 and the sensor 800 are arranged on opposite sides of the torso of the living being, in this case of a human being.

The embodiment depicted at the bottom of FIG. 10 differs from that depicted at the top of FIG. 10 in that the sensor 800 is not integrated into a patient fixation belt 130, but into a cushion, for example, which may be placed onto the patient's rib cage during the operation so as to monitor said patient's breathing. In both embodiments in accordance with FIG. 10, signal transmission by means of load modulation, as was described by means of FIGS. 8a, 8b and 9, is employed.

However, it shall be noted that other data transmission techniques, such as other wireless or wired transmission technologies as have also been set forth above by way of example, may also be employed.

In other words, embodiments of FIG. 10 differ from the previously described embodiments of integration into an automobile only in that in embodiments of FIG. 10, other environment-related integration objects, or first and second means 110, 130, are employed. The measuring principle remains unchanged. Here, the transmitter unit comprising the primary coil 230 is attached underneath the functional bed or operating table 110 opposite the patient's rib cage, and the measuring unit 800 comprising the secondary coil 310 and the load modulation is applied to the patient's rib cage. As was set forth above, there are several application variants of how the measuring unit 800 may be attached to the patient's rib cage. For example, it may be integrated into the patient fixation belt 130 (see top of FIG. 10), or may simply be placed onto the rib cage in the form of a rubber cushion or the like (see bottom of FIG. 10).

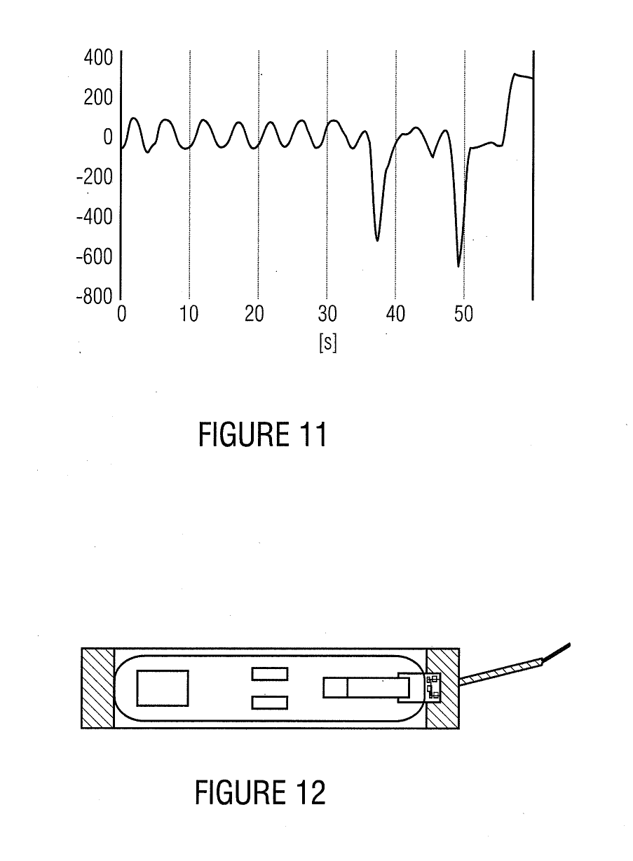

FIG. 11 shows an exemplary diagram of a breathing curve over several seconds. The time, in seconds, is plotted on the x axis, and a measure of the positive and negative change in the distance between the active transmitter and the sensor, or of the negative and positive deviation from a reference distance between the active transmitter and the sensor, is plotted on the y axis.

FIG. 12 shows an example of a prototype of the receiver unit comprising the secondary coil, or the sensor, said prototype comprising integrated adaptation and demodulation electronics.

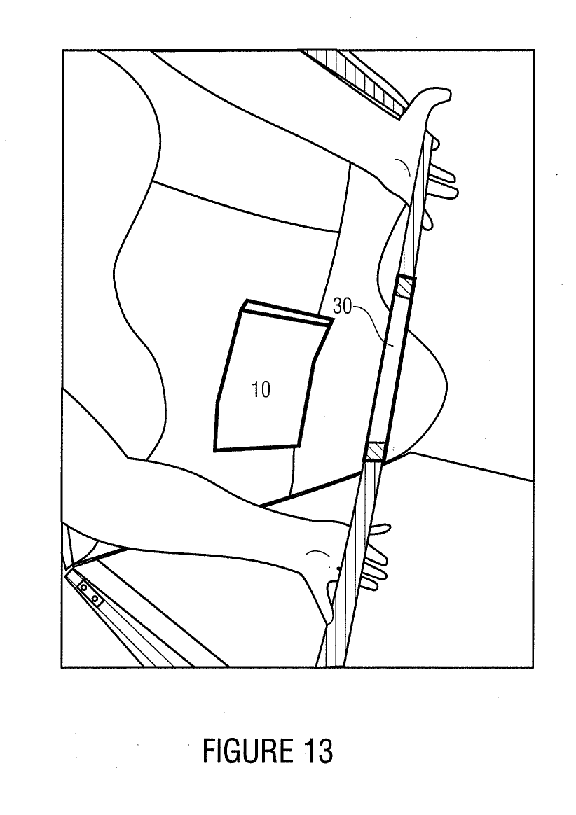

FIG. 13 shows an embodiment of the device for sensing respiration of a living being, wherein the active transmitter 10 is integrated into the backrest of the car seat (see white border) and the sensor, or receiver unit, 30 is integrated into the retention system or safety belt.

It may therefore be stated in summary that embodiments of the present invention realize a “device and method for measuring respiration activity”, further embodiments realize a “device and method for measuring respiration activity by means of loosely inductively coupled coils”, and, yet further embodiments realize a “device and method for measuring respiration activity, breathing rate, breathing amplitude and breathing volume by means of loosely inductively coupled coils”.

Embodiments of the present invention additionally relate to a method and a device for measuring respiration activity which comprise a transmitter device and a receiving device, wherein the receiving device is based on a change in the distance between the transmitter unit and the receiver unit and may therefore be sensed using metrological means. The measurement signals within the receiver may be caused by different physical mutual influences between the transmitter unit and the receiver unit.

Variants of these embodiments further comprise a device and a method for sensing the breathing movements in the torso of a living being by means of inductively coupled coils, breathing movements being recognizable, in the form of positional deviations of the secondary coil relative to the primary coil, in that they cause changes in the induced voltage in the secondary coil.

Further ones of the above-mentioned embodiments provide a device and a method for sensing respiration activity, the breathing rate, the breathing amplitude, and the breathing volume by means of the breathing movements which may be detected in the torso of a living being in the form of positional deviations of the secondary coil from the primary coil.

Further variants of the previously mentioned embodiments relate to a measuring device for sensing the breathing movements in the torso of a living being by means of inductively coupled coils.

Yet further variants of the previously mentioned embodiments of the device provide a measuring device for sensing the breathing movements in the torso of a living being by means of inductively coupled coils (primary coil and secondary coil), the measuring system being equipped with additional measuring coils for stabilizing purposes or for measuring interference effects or for measuring amplitude fluctuations, or in other words, the primary coil system and the secondary coil system comprising additional measuring coils.

Further embodiments of the device provide a measuring system, integrated into the seat and into the safety belt, for sensing respiration activity as a continuous signal. In this context, respiration activity may be measured at at least one, but also at several body parts. To this end, the transmitting and receiving devices are implemented a number of times.

In an alternative variant of the above-mentioned embodiment, a measuring system for sensing respiration activity, which is integrated into a functional bed or an operating table, is provided as a continuous signal. Again, respiration activity may be measured at at least one, but also at several body parts; in the latter case, the transmitting and receiving devices are implemented a number of times.

In a further embodiment, the invention provides a medical system for monitoring the vital parameters of a living being, in particular respiration activity, the breathing rate, the breathing amplitude, and the breathing volume.

Further embodiments of the present invention provide a driver assistance system for medical monitoring of the driver's state of health, in particular respiration activity, the breathing rate, the breathing amplitude, and the breathing volume.

Embodiments further provide a measuring system, integrated into the seat and the safety belt, for sensing respiration activity as a continuous signal.

Further embodiments may also be mounted or integrated in other means or devices.

Instead of inductive coupling, further embodiments comprise electromagnetic coupling between the active transmitter and the sensor. The active transmitter 10 and the sensor 30 may each comprise at least one dipole or patch antenna 230, 310, and be electromagnetically coupled to each other, so that the signal provided depends on a change in the field strength in the sensor, said change in the field strength depending on the change in the distance. Instead of the dipole or patch antenna, the active transmitter and/or the sensor may generally comprise antennas for UHF (ultra-high frequency), micrometer or millimeter wave ranges.

In further embodiments, the transponder or the sensor may be attached directly to the body, e.g. in the form of an adhesive plaster, into which the transponder is integrated, and may thus enable measurement even while the body is turning. Due to the direct contact with the patient's body, an improved effect and/or accuracy of the measurement is enabled, and, additionally, personalization is enabled, e.g. with regard to specific standard values or alarm functions for the patient.

In further embodiments, the sensor or transponder may also be integrated into pieces of clothing.

Thus, the invention relates to both a medical system for monitoring the vital parameters of a person, in particular respiration activity, and to a method and a device for sensing the breathing movements in the body of a living being in general, i.e., for example, of human beings, animals, etc.

The field of application of the embodiments of the present invention lies, for example, in the area of preventive, monitoring and back-up medicine. Direct application is possible, for example, in sensing respiration activity in somnology, sports medicine and home care (monitoring the patient in their homely environment).

Depending on the circumstances, the embodiments of the inventive methods may be implemented in hardware or in software. The implementation may be effected on a digital storage medium, in particular a disc, CD or DVD having electronically readable control signals which cooperate with a programmable computer system such that one of the embodiments of the inventive methods is performed. Generally, the embodiments of the present invention therefore also consist in software program products or computer program products or program products having a program code, stored on a machine-readable carrier, for performing one of the embodiments of the inventive methods, when one of the software program products runs on a computer or on a processor. In other words, an embodiment of the present invention may therefore also be realized as a computer program or software program or program having a program code for performing an embodiment of an inventive method, when the program runs on a processor.

In this context, the processor may be constituted by a computer, a chip card, a digital signal processor, or any other integrated circuit.

While this invention has been described in terms of several embodiments, there are alterations, permutations, and equivalents which fall within the scope of this invention. It should also be noted that there are many alternative ways of implementing the methods and compositions of the present invention. It is therefore intended that the following appended claims be interpreted as including all such alterations, permutations and equivalents as fall within the true spirit and scope of the present invention.

1. A device for sensing respiration of a living being, comprising:
an active transmitter configured to generate a magnetic or electromagnetic field; and
a sensor arranged on the torso of the living being and configured to provide a signal which depends on the magnetic or electromagnetic field and on a change in the distance, caused by the respiration of the living being, between the active transmitter and the sensor.

2. The device as claimed in claim 1, wherein the active transmitter and the sensor each comprise an antenna or an antenna system.

3. The device as claimed in claim 2, wherein the active transmitter and the sensor each comprise at least one antenna coil and are inductively coupled to each other, so that the signal provided depends on a change in the induction within the sensor which is caused by the change in the distance.

4. The device as claimed in claim 2, wherein the active transmitter and the sensor each comprise at least one antenna for UHF (ultra-high frequency), micrometer or millimeter wave ranges and are electromagnetically coupled to each other, so that the signal provided depends on a change in the field strength within the sensor which is caused by the change in the distance.

5. The device as claimed in claim 1, wherein the active transmitter and the sensor are configured to be arranged on opposite sides of the torso of the living being.

6. The device as claimed in claim 1, further comprising:
a first object and a second object;
the first object and the second object being configured and connected to each other in such a manner, the active transmitter being arranged in or on the first object in such a manner, and the sensor being arranged in or on the second object in such a manner, that the active transmitter and the sensor are arranged on opposite sides of the torso, irrespective of a turn of the torso of the living being.

7. The device as claimed in claim 1, wherein the first object or the second object is a backrest of a seat or car seat.

8. The device as claimed in claim 1, wherein the first object or the second object is a lying surface.

9. The device as claimed in claim 7, wherein the second object or the first object is a belt or a safety belt.

10. The device as claimed in claim 1, wherein neither the first object nor the second object is a piece of clothing or is firmly connected to the body.

11. The device as claimed in claim 1, further comprising:
an evaluation unit configured to determine, on the basis of the signal provided, a breathing rate, a breathing amplitude, and/or a breathing volume of the respiration.

12. The device as claimed in claim 11, wherein the sensor and the evaluation unit are connected to each other via a wired or wireless communication interface so as to transmit the provided signal from the sensor to the evaluation unit.

13. The device as claimed in claim 12, wherein the sensor is a transponder, and the wireless communication interface is based on a load modulation method or a back-scattering method.

14. A method of sensing respiration of a living being, comprising:
generating a magnetic or electromagnetic field by means of an active transmitter; and
providing a signal by means of a sensor arranged on the torso of the living being, the signal depending on the magnetic or electromagnetic field and on a change in the distance, caused by the respiration of the living being, between the active transmitter and the sensor.

15. A measuring system for sensing respiration of a person situated within a motor vehicle, comprising:
an active transmitter integrated into a backrest of a car seat of the motor vehicle and configured to generate a magnetic or electromagnetic field; and
a sensor arranged within a safety belt of the motor vehicle, in a state in which the safety belt is fastened, at the height of the person's torso, and configured to provide a signal which depends on the magnetic or electromagnetic field and on a change in the distance, caused by the respiration of the living being, between the active transmitter and the sensor.
